(19) United States
(12) Patent Application Publication
(10) Pub. No.: US 2026/0272447 A1
(43) Pub. Date: Sep. 17, 2026

(54) SUTURE RETRIEVAL DEVICE

(71) Applicant: BOARD OF REGENTS, THE UNIVERSITY OF TEXAS SYSTEM, Austin, TX (US)

(72) Inventors: Steven HENRY, Austin, TX (US); Shyon PARSA, Austin, TX (US)

(73) Assignee: BOARD OF REGENTS, THE UNIVERSITY OF TEXAS SYSTEM, Austin, TX (US)

(21) Appl. No.: 19/108,979

(22) PCT Filed: Sep. 12, 2023
(86) PCT No.: PCT/US2023/073939

§ 371 Date: Mar. 5, 2025

Related U.S. Application Data
(60) Provisional application No. 63/406,414, filed on Sep. 14, 2022.

(51) Int. Cl. A61B 17/04; A61B 17/00; A61B 17/06; A61B 17/34

(52) U.S. Cl. CPC A61B 17/0469; A61B 17/0483; A61B 17/06166; A61B 17/3417; A61B 2017/00876; A61B 2017/3456

(57) ABSTRACT

A device comprising a trocar may include a trocar body having a channel formed therein; a first opening at a first end of the channel, and an end cap at a second end of the channel. The end cap may include an end cap body at least partially enclosing the channel at the second end of the channel, an opening formed in the end cap body at the second end of the channel allowing passage of a mechanical feature through the first opening and through the second opening, and a tongue extending substantially perpendicularly from a surface of the end cap internal to the channel such that the tongue and at least one wall of the channel defines a groove.

### RELATED APPLICATIONS

This application claims priority to U.S. Provisional Patent Application Ser. No. 63/406,414, filed Sep. 14, 2022, which is incorporated by reference herein in its entirety.

### FIELD OF DISCLOSURE

The present disclosure relates in general to methods and systems for passing a suture through human tissue using a suture retrieval device.

### BACKGROUND

Endoscopic surgeries are minimally-invasive surgical procedures in which a surgeon uses an endoscope to view a surgical area internal to the body. Examples of endoscopic surgeries are arthroscopic surgery and laparoscopic surgery.

Arthroscopic surgery is a common, minimally-invasive surgical procedure in which a surgeon uses an arthroscope, an endoscope that is inserted into a joint through a small incision. Joints in which arthroscopy is used include knee joints, hip joints, shoulder joints, wrist joints, spinal joints, and temporomandibular joints. In traditional surgeries, two injection needles, or trocars, may be inserted into a joint on opposite sides of an injury, such as a tear of a tendon or ligament. These trocars may serve as passageways for the surgeon into the location of the injury.

Laparoscopic surgery is another common, minimally-invasive surgical procedure performed in the abdomen, pelvis, or thoracic cavity using small incisions (usually 0.5-1.5 cm) with the aid of a camera. The laparoscope aids diagnosis or therapeutic interventions with a few small cuts in the abdomen

In arthroscopic surgery, a suture may be inserted through one trocar and through one side of the soft tissue tear, so that a probe also known as a suture receiver may be inserted through the other trocar to receive the suture. At this point, the surgeon may tie two ends of the suture using the probes. FIGS. 1A-1D depict such surgical process. As shown in FIG. 1A, a surgeon may insert the ends of two trocars 100 into the surgical space proximate to and on opposite sides of the injury to be repaired. As shown in FIG. 1B, the surgeon may pass a suture 102 through one trocar 100 and pass a probe 104 through the other trocar 100. As shown in FIG. 1C, the surgeon may attempt to retrieve suture 102 with probe 104, and may attempt to secure suture 102 by wrapping the end of suture 102 around a feature of probe 104 and/or tying a knot within suture 102. If successful, as shown in FIG. 1D, the surgeon may pull suture 102 through the injured tissue and trocar 100 using probe 104. Similar approaches for suture retrieval may also be used in laparoscopic and other endoscopic surgeries.

However, such process is often time-consuming and difficult for a surgeon to perform due to the small space in which the suture must be retrieved. Accordingly, improved approaches to suture retrieval are desired.

### SUMMARY

In accordance with the teachings of the present disclosure, the disadvantages and problems associated with existing approaches for performing sutures during a surgical procedure may be reduced or eliminated.

In accordance with embodiments of the present disclosure, a device comprising a trocar may include a trocar body having a channel formed therein; a first opening at a first end of the channel and an end cap at a second end of the channel, the end cap comprising an end cap body at least partially enclosing the channel at the second end of the channel. The device may also include an opening formed in the end cap body at the second end of the channel allowing passage of a mechanical feature through the first opening and through the second opening and a tongue extending substantially perpendicularly from a surface of the end cap internal to the channel such that the tongue and at least one wall of the channel defines a groove.

In accordance with these and other embodiments of the present disclosure, a method for forming a device comprising a trocar may include forming a channel in a trocar body, with a first opening at a first end of the channel, and forming an end cap at a second end of the channel, wherein the end cap comprises an end cap body at least partially enclosing the channel at the second end of the channel, an opening formed in the end cap body at the second end of the channel allowing passage of a mechanical feature through the first opening and through the second opening, and a tongue extending substantially perpendicularly from a surface of the end cap internal to the channel such that the tongue and at least one wall of the channel defines a groove.

In accordance with these and other embodiments of the present disclosure, a method may include inserting a probe into an interior of a channel of a trocar via a first opening of the trocar at a first end of the trocar, positioning a second end of the trocar proximate to a magnetic suture; further inserting the probe into the trocar such that a magnetic tip formed on an extension extending from the probe passes through a second opening of the trocar at the second end of the trocar to place the magnetic tip in sufficient proximity to the magnetic suture such that a magnetic force between the magnetic tip and magnetic suture attracts the magnetic tip suture to the magnetic tip, and retracting the probe within the trocar to pull the magnetic tip and at least a portion of the suture into the interior of the channel of the trocar.

In accordance with these and other embodiments of the present disclosure, a magnetic suture may include a plurality of strands; and ferromagnetic material applied to the plurality of strands such that the magnetic suture is attracted to a magnet.

Technical advantages of the present disclosure may be readily apparent to one having ordinary skill in the art from the figures, description and claims included herein. The objects and advantages of the embodiments will be realized and achieved at least by the elements, features, and combinations particularly pointed out in the claims.

It is to be understood that both the foregoing general description and the following detailed description are examples and explanatory and are not restrictive of the claims set forth in this disclosure.

### BRIEF DESCRIPTION OF THE DRAWINGS

A more complete understanding of the present embodiments and advantages thereof may be acquired by referring to the following description taken in conjunction with the accompanying drawings, in which like reference numbers indicate like features, and wherein:

FIGS. 1A-1D depict selected steps of a process for performing a suture during an arthroscopic surgery, as is known in the art;

FIG. 2A depicts an external side view of a trocar for use in suture retrieval, in accordance with embodiments of the present disclosure;

FIG. 2B depicts an external bottom view of a trocar for use in suture retrieval, in accordance with embodiments of the present disclosure;

FIG. 2C depicts an isometric, see-through view of a cylindrical channel and second opening of the trocar depicted in FIGS. 2A and 2B, in accordance with embodiments of the present disclosure;

FIG. 3A depicts an isometric perspective view of an end cap of the trocar depicted in FIGS. 2A-2C, in accordance with embodiments of the present disclosure;

FIG. 3B depicts another isometric perspective view of an end cap of the trocar depicted in FIGS. 2A-2C, in accordance with embodiments of the present disclosure; and

FIGS. 4A-4D depict selected steps of a process for retrieving a suture during a surgery using the trocar depicted in FIGS. 2A-2C, in accordance with embodiments of the present disclosure.

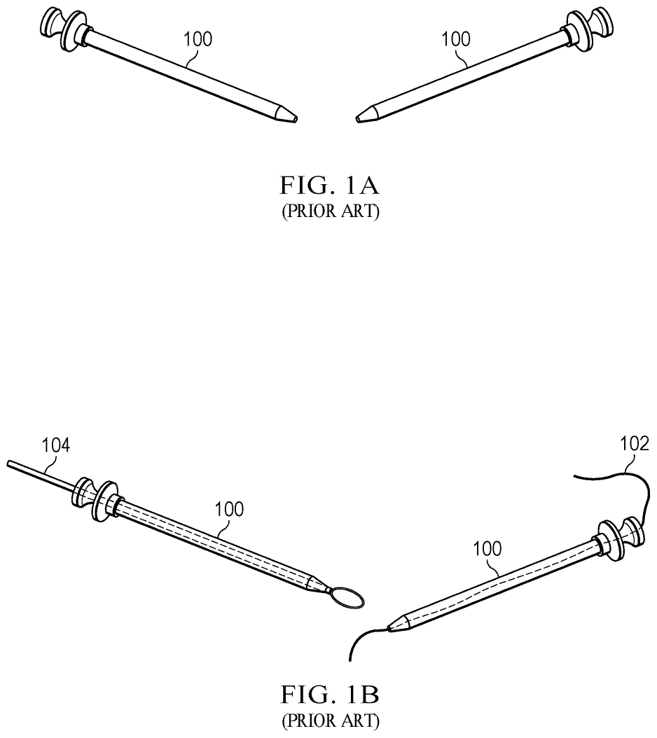

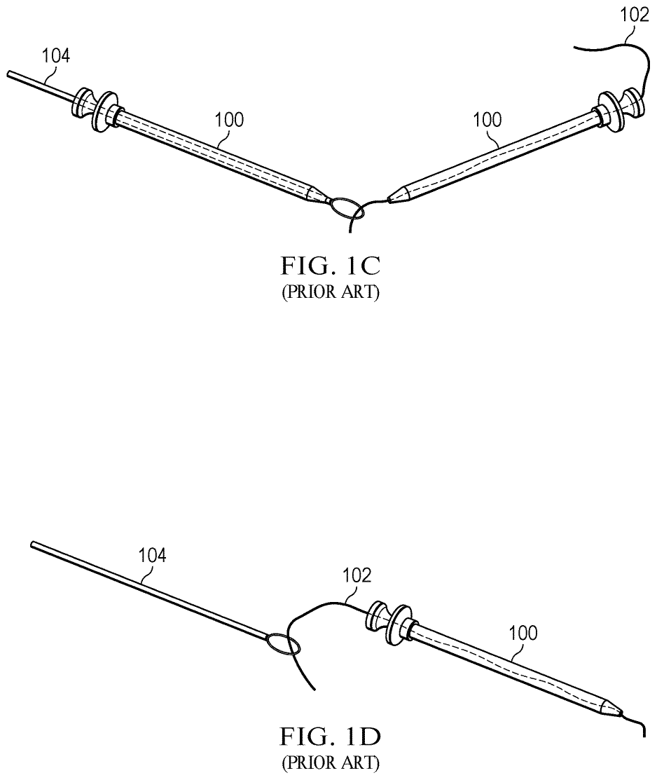

### DETAILED DESCRIPTION

The description below sets forth example embodiments according to this disclosure. Further example embodiments and implementations will be apparent to those having ordinary skill in the art. Further, those having ordinary skill in the art will recognize that various equivalent techniques may be applied in lieu of, or in conjunction with, the embodiment discussed below, and all such equivalents should be deemed as being encompassed by the present disclosure.

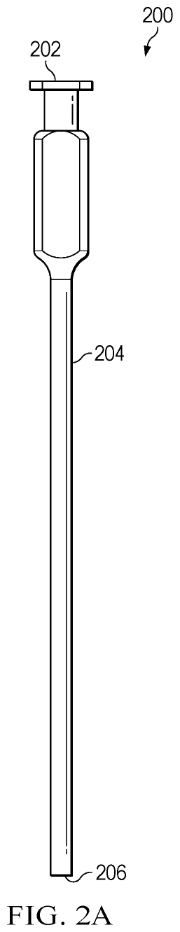

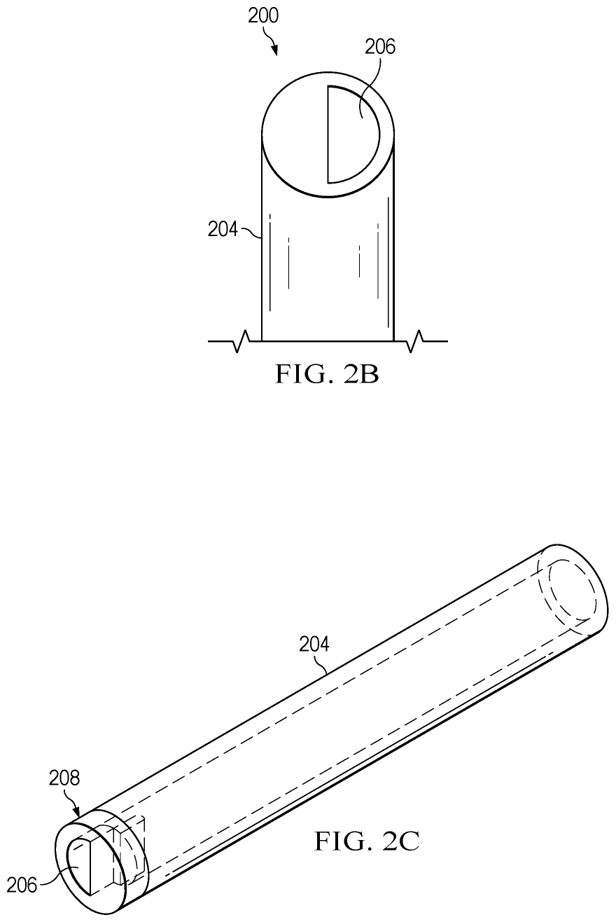

FIG. 2A depicts an external side view of a trocar 200 for use in suture retrieval, in accordance with embodiments of the present disclosure. FIG. 2B depicts an external bottom view of trocar 200, in accordance with embodiments of the present disclosure. FIG. 2C depicts an isometric, see-through view of cylindrical channel 204 and second opening 206 of trocar 200, in accordance with embodiments of the present disclosure. As shown in FIGS. 2A-2C, trocar 200 may have a cylindrical channel 204, first (or top) opening 202 at a first end of cylindrical channel 204, and second (or bottom) opening 206 at a second end of cylindrical channel 204, such that during a surgical procedure, a surgeon may capture a suture external to trocar 200 and retrieve such suture by pulling, with a probe inserted into first opening 202, the suture through second opening 206 and into the interior of cylindrical channel 204. Accordingly, trocar 200 may have a substantially hollow interior for passing such probe and suture. Further, as shown in FIG. 2C, second opening 206 at the end of cylindrical channel 204 may be implemented using an end cap 208 configured to receive and mechanically retain a suture, as described in greater detail below.

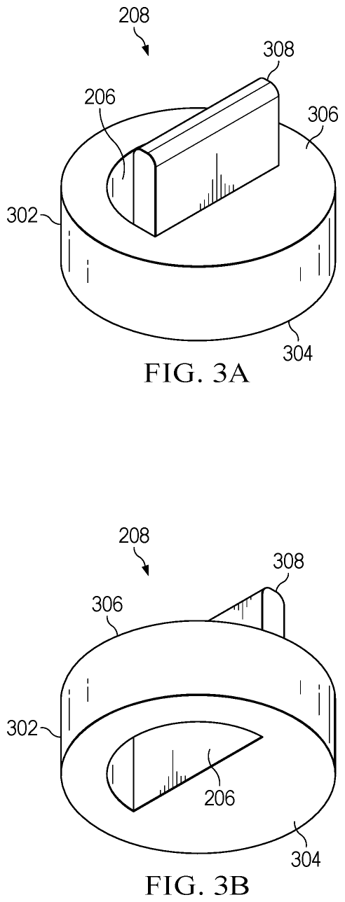

FIG. 3A depicts an isometric perspective view of end cap 208, in accordance with embodiments of the present disclosure. FIG. 3B depicts another isometric perspective view of end cap 208, in accordance with embodiments of the present disclosure. As shown in FIGS. 3A and 3B, end cap 208 may include a substantially solid cylindrical body 302 that at least partially encloses cylindrical channel 204 at the second end of cylindrical channel 204, end cap 208 having second opening 206 formed through body 302. In some embodiments, second opening 206 may be semi-circular in shape and concentric with a circle defining a bottom surface 304 of body 302. A tongue 308 may extend internally to cylindrical channel 204 and extend substantially perpendicularly from a surface of a top 306 of body 302 at a location proximate to second opening 206. In some embodiments, end cap 208 may comprise a structure separate from but mechanically coupled to the main body of trocar 200, whereas in other embodiments, end cap 208 and the main body of trocar 200 may comprise a single piece of material.

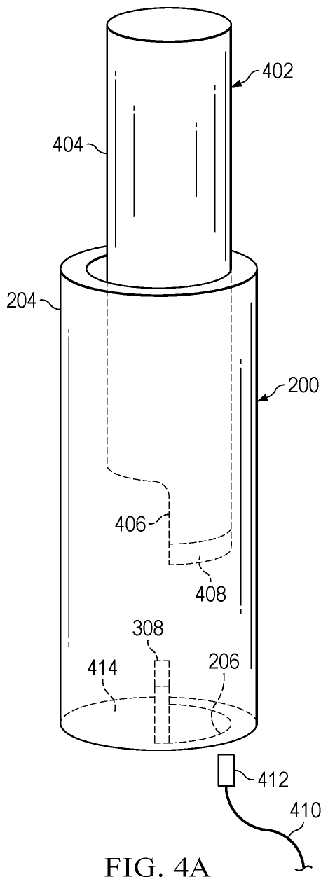

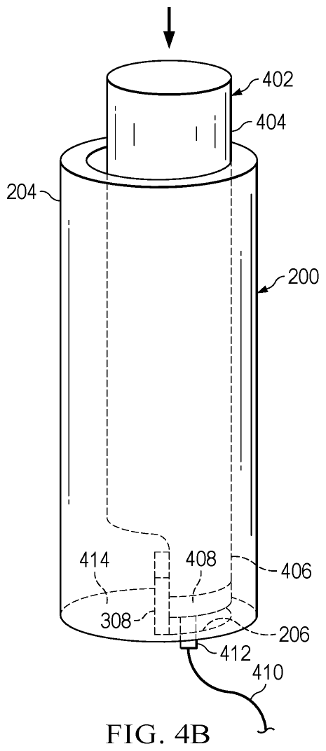

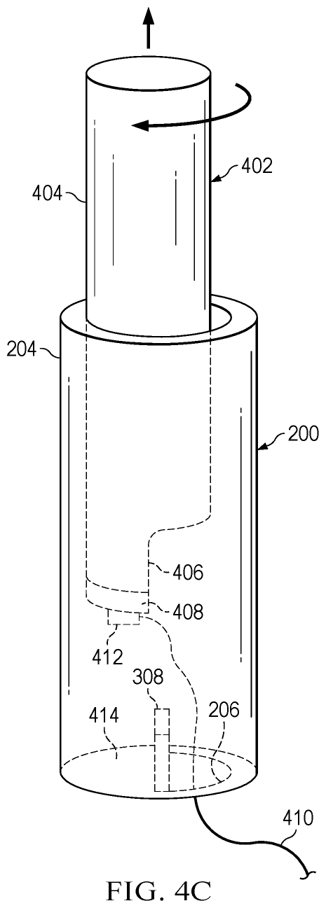

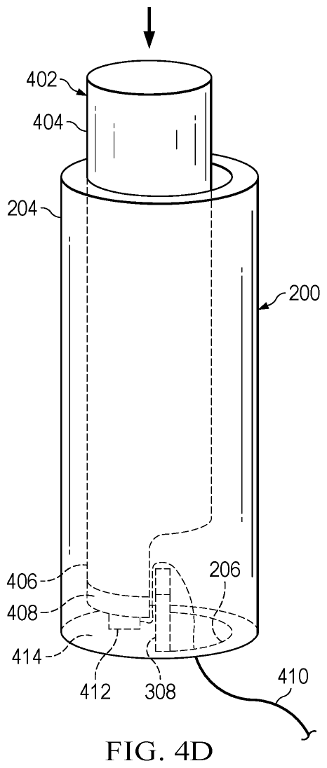

FIGS. 4A-4D depict selected steps of a process for retrieving a suture 410 during a surgery using trocar 200, in accordance with embodiments of the present disclosure. As shown in FIG. 4A, to begin the retrieval process, a surgeon may insert a probe 402 into the interior of cylindrical channel 204 (e.g., via first opening 202, not explicitly shown in FIGS. 4A-4D) and position trocar 200 proximate to suture 410 having a magnetic tip 412. In some embodiments, suture 410 will have been placed in a surgical field via another trocar. Magnetic tip 412 may be formed in any suitable manner, including without limitation application of a magnetic paint (e.g., a paint with small particles of iron dust mixed in) to an end of suture 410, attachment of a magnet to an end of suture 410, or intertwining of magnetic strands with non-magnetic strands of suture 410.

As shown in FIGS. 4A-4D, probe 402 may comprise a body 404 and an extension 406, wherein such extension 406 may have a magnetic tip 408. Body 404 may be substantially cylindrical in shape, with a cylindrical diameter substantially smaller than that of trocar 200, to allow passage of probe 402 into the interior of trocar 200. Extension 406 may be configured (e.g., sized and shaped) to allow passage of magnetic tip 408 through second opening 206. Magnetic tip 408 may comprise any suitable ferromagnetic material, including without limitation neodymium.

As shown in FIG. 4B, to continue the process for retrieving suture 410, the surgeon may insert probe 402 deeper into trocar 200 such that magnetic tip 408 passes through second opening 206 and magnetic tip 408 is placed in sufficient proximity to suture 410 that a magnetic force between magnetic tip 408 and magnetic tip 412 attracts magnetic tip 412 to magnetic tip 408.

Further, as shown in FIG. 4C, the surgeon may partially retract probe 402 within trocar 200 to pull magnetic tip 408 and a portion of suture 410 into the interior of cylindrical channel 204. As also shown in FIG. 4C, the surgeon may then rotate probe 402 relative to cylindrical channel 204 such that magnetic tip 412 and a significant portion of suture 410 pass over a top of tongue 308.

In addition, as shown in FIG. 4D, the surgeon may then insert probe 402 deeper into trocar 200 in order to cause extension 406, magnetic tip 408, magnetic tip 412, and a significant portion of suture 410 to enter into a groove 414 formed by tongue 308 and walls of cylindrical channel 204. By fully inserting extension 406 into groove 414, mechanical forces between probe 402 and interior surfaces of trocar 200 may provide sufficient mechanical force to retain suture 410 within the interior of trocar 200, such that the surgeon may manipulate suture 410 as desired via motion of trocar 200 with suture 410 retained therein.

Although the foregoing contemplates retrieval of a suture 410 by an extension 406 of a probe 402 via magnetic attraction, it is understood that any other suitable approach for mechanically coupling suture 410 to extension 406 may be used in order to retract a significant portion of suture 410 to within the interior space of trocar 200.

Further, although the Background section of this disclosure contemplates suturing in the context of arthroscopic and laparoscopic surgeries, it is understood that the systems and methods described herein may be applied to other endoscopic surgeries and/or to any other suitable type of surgery for which such systems and methods may be beneficial.

Additionally, it is understood that sizes of the various components of trocar 200 described herein, including without limitation first opening 202, cylindrical channel 204, second opening 206, end cap 208, body 302, bottom surface 304, top 306, tongue 308, probe 402, body 404, extension 406, magnetic tip 408, and groove 414 may be adapted to the particular surgical application for which trocar 200 is used. For example, a larger trocar 200 (and larger components of trocar 200) may be desirable for surgeries involving shoulder and knee joints than that which may be desirable for surgeries involving wrist joints. Similarly, it is understood that a size of suture 410 described herein, including without limitation the size of its magnetic tip 412, may be adapted to the particular surgical application for which trocar 200 and suture 410 are used.

Moreover, in addition to application of the systems and methods described herein to surgeries, the systems and methods described herein may be further generally applied to any application in which it may be desirable to mechanically retrieve and mechanically retain any mechanical member analogous to suture 410 within another mechanical member analogous to trocar 200.

As used herein, when two or more elements are referred to as “coupled” to one another, such term indicates that such two or more elements are in mechanical communication, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art, and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

What is claimed is:

1. A device comprising a trocar including:
a trocar body having a channel formed therein;
a first opening at a first end of the channel; and
an end cap at a second end of the channel, the end cap comprising:
an end cap body at least partially enclosing the channel at the second end of the channel;
an opening formed in the end cap body at the second end of the channel allowing passage of a mechanical feature through the first opening and through the second opening; and
a tongue extending substantially perpendicularly from a surface of the end cap internal to the channel such that the tongue and at least one wall of the channel defines a groove.

2. The device of claim 1, further comprising a probe configured to be inserted into the channel of the trocar, the probe comprising:
a probe body;
an extension extending from the probe body; and
a magnetic tip on an end of the extension;
wherein the extension is configured to allow passage of the magnetic tip through the second opening.

3. The device of claim 2, wherein the extension is further configured to allow passage of the magnetic tip into the groove.

4. The device of claim 2, wherein the magnetic tip comprises neodymium.

5. The device of claim 1, wherein the second opening is semicircular in shape.

6. The device of claim 1, wherein the channel is cylindrical in shape.

7. A method for forming a device comprising a trocar including:
forming a channel in a trocar body, with a first opening at a first end of the channel; and
forming an end cap at a second end of the channel, the end cap comprising:
an end cap body at least partially enclosing the channel at the second end of the channel;
an opening formed in the end cap body at the second end of the channel allowing passage of a mechanical feature through the first opening and through the second opening; and
a tongue extending substantially perpendicularly from a surface of the end cap internal to the channel such that the tongue and at least one wall of the channel defines a groove.

8. The method of claim 7, further comprising forming a probe configured to be inserted into the channel of the trocar, wherein forming the probe comprises:
forming an extension extending from a probe body;
forming a magnetic tip on an end of the extension; and
configuring the extension to allow passage of the magnetic tip through the second opening.

9. The method of claim 8, further comprising configuring the extension to allow passage of the magnetic tip into the groove.

10. The method of claim 9, wherein the magnetic tip comprises neodymium.

11. The method of claim 7, further comprising forming the second opening to be semicircular in shape.

12. The method of claim 7, further comprising forming the channel to be cylindrical in shape.

13. A method comprising:
inserting a probe into an interior of a channel of a trocar via a first opening of the trocar at a first end of the trocar;
positioning a second end of the trocar proximate to a magnetic suture;
further inserting the probe into the trocar such that a magnetic tip formed on an extension extending from the probe passes through a second opening of the trocar at the second end of the trocar to place the magnetic tip in sufficient proximity to the magnetic suture such that a magnetic force between the magnetic tip and magnetic suture attracts the magnetic tip suture to the magnetic tip; and
retracting the probe within the trocar to pull the magnetic tip and at least a portion of the suture into the interior of the channel of the trocar.

14. The method of claim 13, further comprising:
rotating the probe relative to the channel to pass at least a portion of the suture over the top of a tongue extending internally to the channel perpendicularly from a surface of an end cap formed at the second end of the trocar, wherein the end cap partially encloses the second end of the trocar and wherein the end cap has the second opening formed therein; and
further inserting the probe into the trocar to cause the magnetic tip and the magnetic suture to enter into a groove formed by the tongue and walls of the channel in order to mechanically retain the magnetic suture within the interior of the trocar.

15. A magnetic suture comprising:
a plurality of strands; and
ferromagnetic material applied to the plurality of strands such that the magnetic suture is attracted to a magnet.

16. The magnetic suture of claim 15, wherein the ferromagnetic material comprises a magnet attached to an end of the magnetic suture.

17. The magnetic suture of claim 15, wherein the ferromagnetic material comprises a magnetic paint applied to an end of the magnetic suture.

18. The magnetic suture of claim 15, wherein the ferromagnetic material comprises a plurality of magnetic strands.
